(19) United States
(12) Patent Application Publication
(10) Pub. No.: US 2023/0147895 A1
(43) Pub. Date: May 11, 2023

(54) SYSTEM AND METHOD FOR CROSS-LANGUAGE SPEECH IMPAIRMENT DETECTION

(71) Applicants: Marzyeh GHASSEMI, Toronto (CA); WINTERLIGHT LABS INC., Toronto (CA)

(72) Inventors: Aparna BALAGOPALAN, Toronto (CA); Jekaterina NOVIKOVA, Toronto (CA); Marzyeh GHASSEMI, Toronto (CA)

(21) Appl. No.: 17/779,493

(22) PCT Filed: Nov. 27, 2020
(86) PCT No.: PCT/CA2020/051637

§ 371 Date: May 24, 2022

Related U.S. Application Data
(60) Provisional application No. 62/941,829, filed on Nov. 28, 2019.

(51) Int. Cl. A61B 5/00; G10L 25/66; G10L 25/30; G10L 15/26

(52) U.S. Cl. CPC A61B 5/4803; A61B 5/4064; A61B 5/7267; G10L 15/26; G10L 25/30; G10L 25/66

(57) ABSTRACT

A system and method for detecting speech impairment employing a machine learning model extends the use of a model trained exclusively in a target language to classify input obtained from a speech sample in a source language different from the target language. Features extracted from a transcript of the speech sample in the source language are subject to a mapping to features in the target language, then provided as input to the model. The mapping is determined using a domain adaptation system implementing an algorithm such as an optimal transport algorithm trained using a healthy speech dataset to map probability distributions of the features from the source to the target language.

### CROSS-REFERENCE TO RELATED APPLICATIONS

This application claims priority to U.S. Provisional Application No. 62/941,829 filed Nov. 28, 2019, the entirety of which is incorporated herein by reference.

### TECHNICAL FIELD

The present disclosure relates to automated detection of speech-based cognitive impairment symptoms, and in particular the use of domain adaptation strategies to extend the use of datasets across languages.

### TECHNICAL BACKGROUND

Generally, datasets of appropriate quality and size are necessary when employing machine learning (ML) in the healthcare domain, due to the high cost of errors and the need to ensure fair and unbiased decision-making.

Studies have considered machine learning based approaches to detection of cognitive impairment or speech impairment, such as aphasia, as a symptom of cognitive impairment. However, these studies have been largely restricted to single-language settings. This is likely due to the dearth of medical speech datasets in multiple languages. As a result, much of the prior work on computational methods for detecting signs of aphasia, for example, focuses on ML models developed for a single language. Where multiple languages or cross-language evaluation have been considered, such studies were limited to non-speech-related features or to feature patterns for a single feature.

### BRIEF DESCRIPTION OF THE DRAWINGS

In drawings which illustrate by way of example only embodiments of the present application,

FIG. 1 is a schematic of a computer-implemented system for cross-language speech impairment detection.

FIG. 2 is a flowchart illustrating a method for classifying speech data in a source language using a classification model developed in a target language, using the system of FIG. 1.

FIG. 3 is a schematic of an example data processing system that may be employed in the system of FIG. 1.

FIG. 4 is a table setting out performance of various optimal transport domain adaptation systems for different classification models, as may be implemented in the system of FIG. 1.

FIG. 5 is a graph illustrating classification performance based on data size.

### DETAILED DESCRIPTION

Multi-language speech datasets are scarce and often have small sample sizes in the medical domain. This has an adverse impact on the development of ML-based approaches to detection of speech impairment as a symptom of certain types of cognitive impairment, particularly in the case of speakers of low-resource languages for which there are only small datasets of examples of impaired speech. As those skilled in the art understand, larger and more diverse are required to produce a ML model with an acceptable error rate—due to the consequences of errors in diagnosis—and to reduce the effect of bias.

To mitigate this problem of data availability, various solutions have been proposed including the creation of novel sources of data, developing data-efficient algorithms, and employing domain adaptation from low-resource domains to resource-rich domains. For instance, Li et al. (Bai Li, Yi-Te Hsu, and Frank Rudzicz. Detecting dementia in Mandarin Chinese using transfer learning from a parallel corpus. In Proceedings of the 2019 Conference of the North American Chapter of the Association for Computational Linguistics: Human Language Technologies, Volume 1 (Long and Short Papers), pages 1991-1997, 2019) used paired samples of movie and television subtitles data in Mandarin and English to train a regression model between independently engineered features for detection of dementia. However, this technique relies on paired data which is not always available, particularly for low-resource languages.

As can be seen from the discussion below, it was surprisingly discovered that out-of-domain, unpaired, single-speaker, healthy speech data may be used effectively to adapt a ML classifier model across languages, using the unpaired healthy speech data to train a mapping between a feature set in a source language (e.g., a low-resource language with sparser in-domain sample data) and in a target language (e.g. a language with richer resources available, with more plentiful in-domain data). This may prove particularly useful in the detection of speech-based cognitive impairment symptoms, because it is more easily generalized not only to other languages, but to other use cases where paired data may not be available to train a ML model.

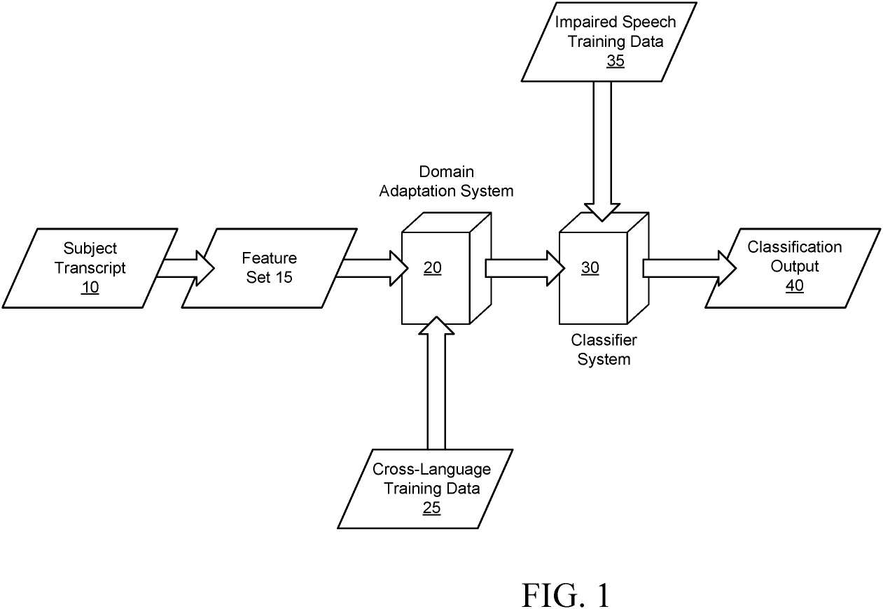

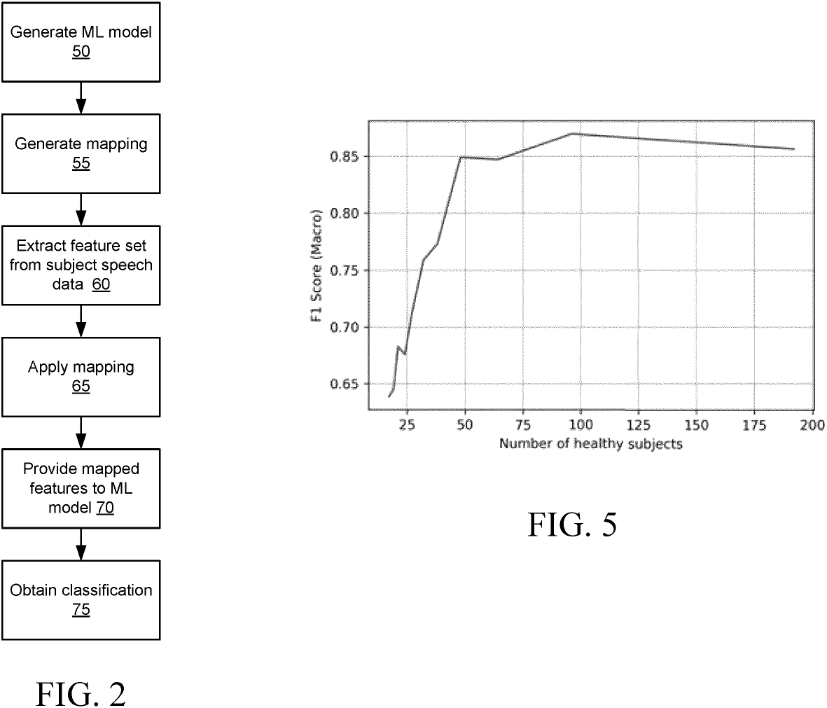

A high-level illustration of the system and process is depicted in FIGS. 1 and 2. Briefly, a ML classifier model is developed using a single-language impaired speech training dataset 35 in the target language (step 50) for execution by a classifier system 30. Additionally, a domain adaptation system 20 is trained using a cross-language training dataset 25, comprising healthy speech data in the source language and a target language, to map or transfer feature sets in the source language to features sets in the target language (step 55). The feature sets employed in training the domain adaptation system are the same feature sets employed in the single-language impaired speech training of the ML classifier.

The healthy speech data may be considered to be out of domain, in that it is not necessarily collected for a medical or diagnostic application; rather, it may be sourced from any suitable corpus providing speech samples in the different languages. As will be seen from the examples below, the cross-language training dataset 25 may also include in-domain, impaired speech data in the source and target languages as well. However, the amount of impaired speech data in the cross-language training dataset 25 may be limited to only a small proportion of the dataset; for instance, less than 10%, and even less than 5%, as can be seen from the examples below. In both the ML classifier and domain adaptation training, both the healthy speech and impaired speech data may be unpaired data, again as can be seen in the examples below.

Input speech data from a speaker in a source language, here in the form of a transcript 10, is featurized to extract a feature set 15 based on linguistic elements of speech (step 60). The feature set 15 is provided as input to a domain adaptation system 20, which maps the input feature set 15 to a mapped feature set (step 65), which is then provided as input to the classifier system 30 executing the ML classifier model (step 70). The classifier system 30 outputs a result 40, classifying the input mapped feature set as healthy speech or impaired speech (step 75). It will be understood by those skilled in the art that the “healthy” speech classification may be considered as a “non-impaired” speech classification—that is to say, speech that is not identified by the classifier system 30 as impaired speech.

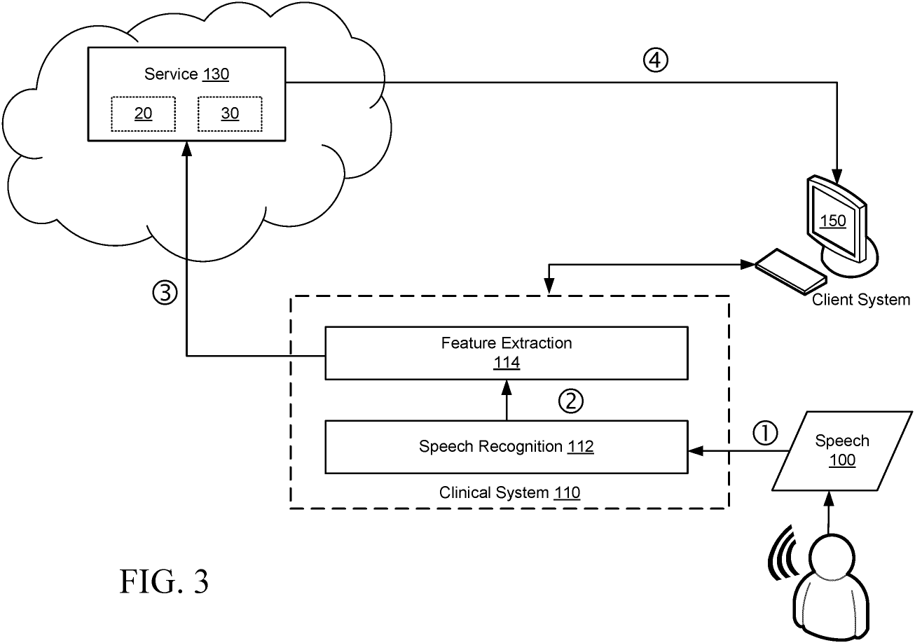

These systems and methods may be advantageously implemented in a data processing system, such as the example system depicted in FIG. 3. In this example, detection of impaired speech is carried out using a cloud-based or otherwise remote analysis service 130 communicating with one or more client systems 150 over a network. Such a remote service 130 is preferably operated in compliance with any applicable privacy legislation, such as the United States Health Insurance Portability and Accountability Act of 1996 (HIPAA).

Individual client systems 150 may be computer systems in clinical or consultation settings, communicating with the remote service 130 over a wide area network (e.g., the Internet). It is contemplated that users may implement clinic or patient management software locally, and that preferably, personally identifying information (PII) of a patient—which can include recorded utterances—is stored securely, and even locally, to avoid accidental dissemination of PII by third-party services. This is reflected in the example data processing system of FIG. 3, in which a patient’s speech is received and recorded 100 using any appropriate recording technology, and provided to a clinical system 110. The clinical system 110 may comprise the clinic or patient management software, or a dedicated application that communicates with the remote analysis service 130. The clinical system 110 may comprise a further remote server system or cloud-based system, accessible by the client system 150 via a network. The clinical system 110 may be operated or hosted by a third party provider, but in some implementations, it may be hosted locally in the clinical setting, e.g., co-located with the client system 150.

The clinical system 110 in this example is configured to receive the recorded speech (1), convert the recorded speech to text using a suitable speech recognition module 112 as would be known to those skilled in the art, and to provide the recognized speech (2) to a feature extraction module 114 which is configured to recognize the linguistic features of interest for use in classification, and to generate the feature set that will be used as input to a ML classifier system. In some implementations, a transcript of the subject’s speech may be produced manually, and the featurization may be carried out manually as well. The generated feature set is then provided (3) to the remote analysis service 130; this feature set data may be provided to the remote analysis service 130 anonymously, for example identified only using a patient identification number.

The remote analysis service 130 may implement both the domain adaptation system 20 and the classifier system 30 described with reference to FIG. 1. It will be appreciated, though, that actual training of the domain adaptation system 20 and classifier system 30 may be carried out outside the illustrated data processing system, with the resultant model and mapping imported into the remote analysis service 130 for execution. The remote analysis service 130 provides the received feature set to the domain adaptation system 20 to obtain the mapped feature set, and the mapped feature set is then applied as input to the classifier system 30 to generate a classification result, which is then transmitted (4) over the network to the client system 150.

The various services and systems described above may be implemented using networked computer systems. The configuration of such servers and systems, including required processors and memory, operating systems, network communication subsystems, and the like, will be known to those skilled in the art. Communications between various components and elements of the data processing system may occur over private or public connections, preferably with adequate security safeguards as are known in the art. In particular, if communications take place over a public network such as the Internet, suitable encryption is employed to safeguard the privacy of data exchanged between the various components of the data processing system.

In the implementation and examples described below, the domain adaptation system was trained to effect cross-language transfer of an aphasic speech detection ML model trained in English (a resource-rich domain, with a larger available set of aphasic speech samples) to lower-resource languages, namely French and Mandarin. Aphasia is a form of speech impairment that affects speech production and/or comprehension. It typically occurs due to brain injury, most commonly from a stroke. Evaluation of speech is an important part of diagnosing aphasia and identifying sub-types. Aphasic speech exhibits several common patterns; e.g., omitting short words (“a”, “is”), using made-up words, etc. While it has been shown to be possible to detect aphasic speech using ML from patterns of linguistic features in spontaneous speech, the prior art has mostly been restricted to a single language, as noted above.

In implementation, it was found that by featurizing speech to identify distributions of linguistic elements in speech, and by employing domain adaptation to correlate or map the probability distribution of the linguistic elements from a source language to a target language, ML models developed using robust data in the target language could be effectively used to classify speech as healthy or impaired (in the examples, healthy or aphasic) in a source language, particularly when the source and target languages are significantly different (e.g., English versus Mandarin). In the examples described below, various optimal transport (OT) systems were employed. It was also found that in featurizing speech using distributions of linguistic elements of speech, rather than the linguistic elements themselves, the need for paired data to train either the ML classifier models or the domain adaptation systems was avoided or reduced. In these examples, distributions of selected parts of speech (POS) were employed as the feature set, as described below.

### Data Sources

The use of different OT domain adaptation strategies was tested for the detection of aphasic speech in French and Mandarin speakers. A first set of speech datasets were obtained from AphasiaBank, a database of multimedia interactions for the study of aphasic communication in aphasia maintained by the TalkBank Project and available at aphasia.talkbank.org (Brian MacWhinney, Davida Fromm, Margaret Forbes, and Audrey Holland. Aphasiabank: Methods for studying discourse. Aphasiology, 25(11):1286-1307, 2011.) In the dataset, aphasic speakers have various subtypes of aphasia including:
Broca aphasia or non-fluent aphasia: Individuals with Broca’s aphasia have trouble speaking fluently but their comprehension can be relatively preserved.
Wernicke’s aphasia or fluent aphasia: In this form of aphasia the ability to grasp the meaning of spoken words is chiefly impaired, while the ease of producing connected speech is not much affected.
Anomic aphasia: Individuals with anomic aphasia can understand speech and read well but frequently are unable to obtain words specific to what they wish to talk about -particularly nouns and verbs.
Transcortical aphasia: Individuals with this type of aphasia have reduced speech output, typically due to a stroke.
Conduction aphasia: Individuals with can comprehend speech and read well, but have significant difficulty in repeating phrases.

In these datasets, all participants perform multiple speech-based tasks, such as describing pictures, story-telling, free speech and discourse. All such tasks were manually transcribed into single transcripts for analysis of OT domain adaptation systems, following the CHAT protocol (Nan Bernstein Ratner. Brian MacWhinney, The Childes Project: Tools for analyzing talk. Hillsdale, NJ: Erlbaum, 1991. pp. xi+ 360. Language in Society, 22(2):307-313, 1993.) The speech samples from AphasiaBank were classified into two classes, healthy and aphasic, where constitutes all subtypes mentioned above, using extracted linguistic features. The AphasiaBank transcripts thus constitute unpaired data, in that there are no corresponding transcripts in different languages for a single speaker; nor are there corresponding healthy and aphasic transcripts for a single speaker.

A larger dataset of multi-lingual transcripts, in English, French, and Mandarin, was used to train the domain adaptation systems. These transcripts were obtained from a dataset of TED talks (Jörg Tiedemann. Parallel data, tools and interfaces in opus. In Lrec, volume 2012, pages 2214-2218, 2012.) In total, there were recordings available for 1178 talks, with various speaker accents and styles. To remove potential bias of the domain adaptation system by paired data, overlap between speech transcripts of English and French/Mandarin was removed by dividing the talks into two sets and ensuring that the English transcripts for training the domain adaptation systems were obtained from the first set, while those for French/Mandarin were obtained from the second set. Further, experiments were performed to validate that this choice did not significantly affect results. It was found that using either fully paired or fully unpaired (as described here) data yielded statistically insignificantly different results.

Additionally, similar to the methodology of Li et al. (2019) a larger dataset was created by dividing each narration into segments by considering 25 consecutive utterances (in this case, sentences or fully-coherent units of speech) as one segment, to produce a larger number of transcripts. This number was chosen to maximize the data available because it was observed that the features stabilized with this number of utterances. Differences in values of the eight parts of speech (POS) features (nouns, verbs, subordinating conjunctions, adjectives, adverbs, coordinating conjunctions, determiners, and pronouns, using definitions provided by Universal Dependencies, a collaborative project available at universaldependencies.org) taken from speech samples were compared, for transcript lengths of 5, 25, 50, 75 and 100 utterances each. t-tests were computed between the features and it was found that while 5 out of 8 features were significantly different between transcript lengths of 5 and 25, they stabilize for lengths greater than or equal to 25, i.e., no significant difference between lengths of 25 and 50 (lowest p-value is 0.22), 50 and 75 (lowest p-value is 0.32) and 75 and 100 (lowest p-value is 0.59).

The statistics for the samples used in each language are shown in Table 1 below:

TABLE 1

Number of samples from AphasiaBank and the TED Talks corpus.

| Corpus | Language | Healthy samples | Aphasic samples |
|---|---|---|---|
| AphasiaBank | English | 246 (192) | 428 (301) |
| AphasiaBank | French | 13 (13) | 11 (11) |
| AphasiaBank | Mandarin | 42 (40) | 18 (15) |
| TED Talks | English | 2875 (589) | - |
| TED Talks | French | 2976 (589) | - |
| TED Talks | Mandarin | 2742 (589) | - |

In Table 1, the number of participants (speakers) is indicated in parentheses. Pre-processing and feature extraction

Since the transcripts provided in AphasiaBank include annotations for features such as repetitions, markers for incorrect word usage, etc., it was necessary to remove these annotations prior to use. The pylangacq library (Jackson L. Lee, Ross Burkholder, Gallagher B. Flinn, and Emily R. Coppess. Working with chat transcripts in python. Technical Report TR-2016-02, Department of Computer Science, University of Chicago, 2016.) was used for this purpose due to its capabilities of handling CHAT transcripts. Additional pre-processing steps included stripping the various utterances of punctuations before POS-tagging.

The proportion of the eight POS identified above were extracted over the whole of each individual transcript. Although aphasic speakers perform one additional speech task (where they provide details regarding their stroke) more than control speakers in AphasiaBank, these eight features are agnostic to total length and content of transcripts, and rely more on the sentence complexity. These simple features were used because they are general and have been identified to be important in prior work (Kathleen C Fraser, Frank Rudzicz, and Elizabeth Rochon. Using text and acoustic features to diagnose progressive aphasia and its subtypes. In INTERSPEECH, pages 2177-2181, 2013; Li et al. (2019); S Law, A Kong, L Lai, and C Lai. Production of nouns and verbs in picture naming and narrative tasks by Chinese speakers with aphasia. Procedia, social and behavioral sciences, 94, 2013) across languages. These features are extracted from all languages in the AphasiaBank samples.

To analyse the variance in features across languages, it was determined whether they differ significantly between healthy and aphasic speakers across languages. It was observed that every feature varies significantly between healthy and aphasic speakers of English and Mandarin. It was therefore expected that raw, non-adapted cross-language transfer of models trained on English speech to Mandarin would lead to low performance. Table 2 sets out significant p-values corresponding to t-tests of the eight POS features between English and other languages (after Bonferroni correction). In Table 2, an asterisk (*) indicates the feature is significantly different for both Mandarin and French; a plus symbol (+) indicates the feature is only significantly different between English and Mandarin; and a hash symbol (#) ‘#’ indicates the feature is only significantly different between English and French. A dash (-) indicates there is no significant difference.

TABLE 2

Number of samples from AphasiaBank and the TED Talks corpus.

| | | |
|---|---|---|
| POS/Feature | Aphasia | Control |
| Nouns | + | + |
| Verbs | * | * |
| Subordinating conjunctions | * | * |
| Adjectives | + | * |
| Adverbs | * | * |
| Co-ordinating Conjunctions | + | * |
| Determiners | + | * |
| Pronouns | + | + |

### Establishment of Unilingal and Multilingual Baselines

Due to the lack of baselines on the multilingual AphasiaBank dataset in prior work, new unilingual and multilingual baselines were established as follows:
Unilingual Training: Unilingual baselines were identified for each language using 10-fold cross-validation (CV), stratified by subject so that each subject’s samples did not occur in both training and testing sets in each fold. It was expected that this would provide a lower bound on performance for French and Mandarin AphasiaBank, since it is likely, given the small size of the dataset, that models would underfit and have low generalizable performance across subjects.
Feature Transfer from English with raw, non-adapted features: Transfer baselines were also identified, in which models trained on English AphasiaBank transcripts were evaluated on other languages with no fine-tuning. It was expected that this baseline would be more performant than the unilingual baseline, at least amongst the more-similar Romance languages of French and English, since it utilizes the comparatively larger dataset of English AphasiaBank for training.
Multilanguage Embedding with an Autoencoder. A common representation was obtained for all three languages by encoding the linguistic features using a high capacity autoencoder. This autoencoder, trained on English, French and Mandarin TED Talks datasets (unpaired), maps linguistic features extracted from multilingual transcripts into a shared latent space. The autoencoder consists of 4 hidden layers (2 hidden layers in encoder and decoder respectively) with 5, 3, 3 and 5 units each for the following evaluation of OT training regimes. Hyperparameters were set using a 90-10 train-dev split of samples from each language. All ML classifiers were then trained on the encoded versions of English AphasiaBank and tested on encoded versions of French and Mandarin AphasiaBank. Comparison of other training regimes to this baseline would determine if learning a shared representation across multiple languages is better than OT.

### Selection and Training of Target Language Classification Models

Machine learning models were trained only on the English AphasiaBank (unpaired) dataset for classifying transcripts as healthy or aphasic.

Hyperparameters for the classification models were tuned using grid search with 10-fold cross validation on the English AphasiaBank training set across all settings. Three classifiers were employed for the cross-linguistic classification task: support-vector machine (SVM) with a radial basis function (RBF) kernel, with regularization parameter C = 0.1 and y = 0.001; Random Forest (RF) with 200 decision trees with a maximum depth of 2; and Neural Network (NN) with 2 hidden layers of 100 units each (Fabian Pedregosa, Gaël Varoquaux, Alexandre Gramfort, Vincent Michel, Bertrand Thirion, Olivier Grisel, Mathieu Blondel, Peter Prettenhofer, Ron Weiss, Vincent Dubourg, et al. Scikit-learn: Machine learning in Python. Journal of machine learning research, 12(Oct): 2825-2830, 2011). Since the training set as between the target English language and source French/Mandarin languages was highly imbalanced (see the sample statistics in Table 1), the minority class was oversampled synthetically with the Synthetic Minority Oversampling Technique (SMOTE) (Nitesh V Chawla, Kevin W Bowyer, Lawrence O Hall, and W Philip Kegelmeyer. Smote: synthetic minority over-sampling technique. Journal of artificial intelligence research, 16:321-357, 2002) with k = 3. Prior to oversampling, the training set was normalized and scaled using the median and interquartile range, a common mechanism to center and scale-normalize data which is robust to outliers (Pedregosa et al. (2011)). The same median and interquartile (obtained from the training set) bounds were used to scale the evaluation set in each case.

### Selection and Training of Optimal Transport Systems

OT consists of finding the best transport strategy from one probability distribution function (PDF) to another. This is done by minimizing the total cost of transporting a sample from the source to that in the target. Thus, there needs to be a metric to quantify the different distances between samples in the two probability distributions, as well as solvers to solve the optimization problem of minimizing the total cost of transport, where cost is related to distance between source and target. OT was selected for domain adaptation because the same features are extracted across languages, although their distributions (in terms of feature values, e.g. proportion of nouns) vary from one language to another.

Two OT training regimes were evaluated for both French and Mandarin, across three variants (solvers and distance functions) of OT. The selected variants of OT were as follows:
Earth Movers Distance OT (“OT-EMD”): The Earth Movers Distance or Wasserstein distance between the two distributions is minimized using an optimal transport Network Flow (Nicolas Bonneel, Michiel Van De Panne, Sylvain Paris, and Wolfgang Heidrich. Displacement interpolation using lagrangian mass transport. In ACM Transactions on Graphics (TOG), volume 30, page 158. ACM, 2011).
Gaussian Optimal Transport Mapping (“OT-Gaussian”): The Earth Movers Distance or Wasserstein distance between the two distributions is minimized in the same manner as above. However, the transport map is approximated with a Gaussian kernelized mapping to obtain smoother transport maps (Michael Perrot, Nicolas Courty, R’emi Flamary, and Amaury Habrard. Mapping estimation for discrete optimal transport. In Advances in Neural Information Processing Systems, pages 4197-4205, 2016).
Entropic Regularization OT solver (“OT-EMD-R”): The optimal transportation problem with EMD regularized by an entropic term, turning the linear program into a strictly convex problem that can be solved with the Sinkhorn-Knopp matrix scaling algorithm (Richard Sinkhorn and Paul Knopp. Concerning nonnegative matrices and doubly stochastic matrices. Pacific Journal of Mathematics, 21(2):343-348, 1967). The linear solver proposed by Cuturi (Marco Cuturi. Sinkhorn distances: Lightspeed computation of optimal transport. In Advances in neural information processing systems, pages 2292-2300, 2013) is used.

The PythonOT open source implementation of these algorithms was used (Remi Flamary and Nicolas Courty. Pot python optimal transport library, 2017, available at github.com/PythonOT/POT).

For OT-EMD, the method proposed by Ferradans et al. (Sira Ferradans, Nicolas Papadakis, Gabriel Peyr’e, and Jean-Fran,cois Aujol. Regularized discrete optimal transport. SIAM Journal on Imaging Sciences, 7(3):1853-1882, 2014) was used for out of sample mapping to apply to transport samples from a domain into the other, with other default parameters in the open source implementations. For OT-EMD-R, entropic regularization parameter was set to 3 with all other parameters default. For OT-Gaussian, the weight for linear OT loss was set to 1, and maximum iterations was set to 20, with a stop threshold for iterations set to 1E-05 with the other default parameters in the open source implementation.

OT mappings from each source language (French/Mandarin) to the target language (English) were learned for each algorithm, trained on language pairs (English-French/English-Mandarin) from the TED Talks (unpaired) dataset, according to two training regimes:
Feature Transfer from English with OT domain adaptation, with TED Talks for OT: Classification models were trained on the English AphasiaBank transcripts, then evaluated on other languages with OT adaptation (OT-EMD, OT-EMD-R, OT-Gaussian) with no fine-tuning. The OT models were trained only on the multi-language unpaired TED Talks dataset, i.e, with no aphasic data.
Feature Transfer from English with OT domain adaptation, with TED Talks and AphasiaBank for OT: Classification models were trained on the English AphasiaBank transcripts, then evaluated on other languages OT adaptation (with OT-EMD, OT-EMD-R, OT-Gaussian) with no fine-tuning. In this case, the OT models were trained on the multi-language TED Talks dataset and multi- language AphasiaBank data, i.e, with aphasic data. In this case, half of the aphasic dataset was included with the TED Talks dataset, such that about 3-4% of the OT training dataset consisted of aphasic data.

Overlap in the proportion of AphasiaBank used for learning OT mappings and for evaluating the classifiers was avoided by employing 2-fold cross-validation where one fold is included in the training set for OT and another for evaluation. Since OT involves source and target domain probability estimation, it was expected that adding in-domain data, particularly that of speech-impaired participants, would improve results significantly.

Additionally, it was hypothesized that there would be an observable effect of speech diversity in terms of accents in the OT training set, since the literature shows that accents can have a significant effect on POS features (Elin Runnqvist, Tamar H Gollan, Albert Costa, and Victor S Ferreira. A disadvantage in bilingual sentence production modulated by syntactic frequency and similarity across languages. Cognition, 2(129):256-263, 2013). To study this effect, accents were manually annotated for the presence or absence of a North American accent in the English TED Talks dataset. The TED Talks dataset covers a wide speaker demographic, in terms of sex, age and accents. This was accomplished by an annotator listening to the audio associated with each TED Talk and annotating if the accent is ‘North American’ (NA) or ‘other’. In cases where the accent was not clear, publicly available information regarding nationality of speaker was referenced. In total in the English TED Talks dataset, there were 373 NA accented and 215 ‘other’ accented talks. The impact of increasing accent diversity in training OT algorithms while keeping the size of the dataset constant was evaluated, as discussed in the results below.

### Evaluation of Task Performance

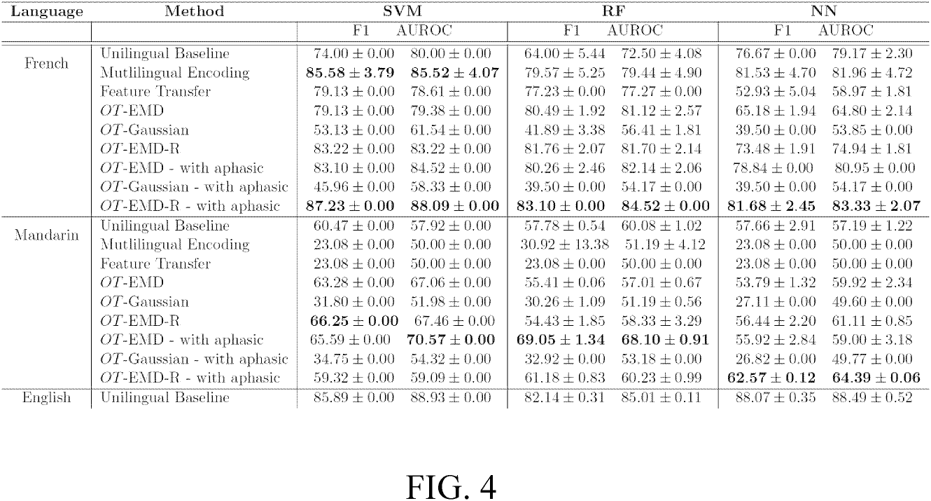

Task performance was evaluated primarily using macro-averaged F1 scores, a measure that is known to be robust to class imbalance. Area under the receiver operating characteristic (AUROC) scores were also determined, since they are often used as a general measure of performance irrespective of any particular threshold or operating point. FIG. 4 sets out the F1 macro and AUROC mean and standard deviation scores across languages for different model settings in OT training, averaged across multiple runs. The results are discussed in further detail below.

It may be noted that zero standard deviations occur when a single class was predicted or when standard deviation < 0.01. The standard deviations were an artifact of the small sample sizes in the evaluation set (24 and 60 for French and Mandarin respectively). Highest F1 scores are shown in bold in FIG. 4 for each language and classifier. Overall, the highest mean F1 scores were obtained with the EMD variants of the OT domain adaptation system, with aphasic samples included in OT training, along with the multilingual TED Talks dataset. However, it can be appreciated from the results that improvements were realized even without the use of aphasic samples in OT training.

### Baseline Results

Baseline performance was found to vary significantly between languages, as can be seen in FIG. 4. For French, using a multilingual encoding or direct feature transfer was found to offer significant improvements over unilingual training, yielding a maximal lift of 15 for RF mean F1, and achieving maximal overall classifier performance using multilingual encoding with a SVM model. In general for French, the multilingual encoding outperformed the feature transfer baseline, but both improve on unilingual results.

For Mandarin, these results were very different; either baseline approach to adaption hurts overall performance as compared to a unilingual baseline, often yielding solutions for which the model would predict just a single class exclusively.

The difference in outcomes for English to French domain adaptation versus English to Mandarin was expected, as noted above. English and French have relatively similar grammatical patterns (e.g., subject, verb, object ordering), whereas Mandarin and English have a number of significant differences (e.g., reduplication, where a syllable or word is repeated to produce a modified meaning).

### OT Variant Results

Among the OT variants (OT-EMD, OT-Gaussian, OT-EMD-R), there was generally stronger performance as compared to the unilingual models and baseline adaptation systems as well. In all but one case, the best-performing OT variant for a given model/language yielded a statistically significant improvement over the best baseline model according to a paired t-test, the notable exception being for the SVM model on French text, which did not achieve statistical significance.

In general, EMD variants of OT (including both OT-EMD-R and OT-EMD) were found to perform better than the Gaussian variant.

### Effect of Aphasic Samples in OT Training

As can be seen from FIG. 4, even without the inclusion of aphasic samples in OT training, the various OT variants work on par with the multilingual baseline with a high-capacity autoencoder, and work significantly better for dissimilar languages (e.g., Mandarin to English), even when the training set used to develop the OT mapping did not include aphasic samples, particularly with the EMD-based OT variants. With the addition of aphasic samples in even a small proportion (3-4%), a strong positive effect is generally seen, with the highest mean F1-score for cross-language classification on the evaluation set increasing to 87.23 (OT-EMD-R with SVM) for French and 69.04 (OT-EMD with SVM) for Mandarin from 83.22 and 66.25 respectively (both significant increases, with p < 0.001 and p = 0.015 respectively).

### Statistically Insignificant Effect of Paired Data

As noted above, the use of paired data was compared to the use of unpaired data, and it was found that the use of paired data in training the OT variants did not significantly improve performance for classification of French or Mandarin samples, over classification using OT variants trained using unpaired datasets. Table 3 below sets out F1-scores, which may be compared with the results of FIG. 4:

TABLE 3

F1 macro scores across languages with OT, with paired data.

| Language | Method | SVM | RF | NN |
|---|---|---|---|---|
| | | F1 | F1 | F1 |
| French | OT-EMD | 83.22 ± 0.00 | 84.71 ± 1.95 | 67.22 ± 2.65 |
| | OT-Gaussian | 46.67 ± 0.00 | 41.89 ± 3.38 | 34.12 ± 3.80 |
| | OT-EMD-R | 83.22 ± 0.00 | 78.84 ± 0.00 | 74.97 ± 3.41 |
| Mandarin | OT-EMD | 59.28 ± 0.00 | 55.35 ± 3.38 | 49.30 ± 2.42 |
| | OT-Gaussian | 25.97 ± 0.00 | 25.53 ± 0.63 | 24.64 ± 0.00 |
| | OT-EMD-R | 60.11 ± 0.00 | 49.25 ± 1.26 | 56.12 ± 2.01 |

### Effect of Data Size on Unilingual Performance

To study the impact of data size on the aphasia detection task, an ablation study was performed in which the size of the English AphasiaBank dataset was artificially reduced by integer factors, while keeping the relative proportion of healthy and aphasic subjects the same. A 10-fold cross-validation was performed for a SVM classifier, with progressively less data. As can be seen in FIG. 5, speech transcripts from at least 50 healthy subjects were required for the classification performance to stabilize, given the feature set. F1 scores (micro and macro) increased non-linearly with the addition of data.

### Speech Diversity Results

As described above, the effect of speech diversity, as measured by the frequency of various accents (NA vs other) in the speech data, on the performance of the various domain adaptation systems was evaluated. Table 4 below sets out the effect of speech diversity on the OT-EMD-R variant for SVM models in French and Mandarin.

TABLE 4

Effect of speech diversity OT-EMD-R implementations.

| Language | Method | OT Dataset Size | SVM | |
|---|---|---|---|---|
| | | | F1 | AUROC |
| French | OT-EMD-R | 286 NA | 83.22 | 83.22 |
| | OT-EMD-R | 215 NA, 71 not NA | 83.22 | 83.22 |
| | OT-EMD-R | 143 NA, 143 not NA | 83.22 | 83.22 |
| | OT-EMD-R | 71 NA, 215 not NA | 87.48 | 87.76 |
| Mandarin | OT-EMD-R | 286 NA | 66.25 | 67.46 |
| | OT-EMD-R | 215 NA, 71 not NA | 62.50 | 63.49 |
| | OT-EMD-R | 143 NA, 143 not NA | 68.51 | 70.24 |
| | OT-EMD-R | 71 NA, 215 not NA | 69.19 | 71.83 |

As can be seen above, for both French and Mandarin, increasing the prevalence of non-North American accents in the OT domain adaption task was found to improve downstream aphasia/non-aphasia classification performance by several F1 points (yielding a score of 87.48 for French and 69.19 for Mandarin). However, these results are not necessarily statistically significantly different than the best results set out in FIG. 4.

The foregoing results demonstrate that POS features extracted from speech transcripts from different languages can be successfully mapped to a target language to aid in medical speech classification tasks, despite any unavailability of paired data. In particular, in comparison to a multilingual baseline with a high-capacity autoencoder, it was found that domain adaption strategies, in particular OT-based domain adaption, can help enable strong predictive models for aphasia detection in low-resource languages that work on par for similar, and significantly better for dissimilar languages. Further, the foregoing results demonstrate that simple-to-extract text-based features—in this example, POS proportions—can be successfully transferred across languages to facilitate detection of speech impairment as a symptom of cognitive impairment and specifically to improve aphasia detection in a cross-language evaluation setting.

While the examples above are mainly focused on optimal transport methods of domain adaptation, it is reasonable to expect from the demonstrated success of this technique that other state of the art domain adaptation techniques such as adversarial domain adaptation (Eric Tzeng, Judy Hoffman, Kate Saenko, and Trevor Darrell. Adversarial discriminative domain adaptation. In Proceedings of the IEEE Conference on Computer Vision and Pattern Recognition, pages 7167-7176, 2017) may likewise prove useful in cross-language transfer of ML models for detection of speech impairment as a symptom of cognitive impairment, and particularly aphasia.

Thus, in accordance with the examples and embodiments discussed above, there is provided a method of detecting speech impairment indicative of cognitive impairment in a subject, the method comprising obtaining a feature set from a subject’s speech data in a source language; applying a mapping to the extracted feature set to provide mapped features, wherein the mapping is defined by a domain adaptation system trained on a domain adaptation dataset comprising healthy speech data in the source language and a target language; providing the mapped features as input to a classifier trained in the target language to classify impaired speech and healthy speech; and obtaining a classification of the subject’s speech data as impaired speech or healthy speech from the classifier.

There is also provided a method of detecting speech impairment indicative of cognitive impairment from a speech sample, the method comprising generating a machine learning model for classifying input as impaired speech or healthy speech, the machine learning model trained on a first dataset in a target language, the first dataset comprising impaired speech data and healthy speech data; generating an optimal transport mapping of a feature set employed in the machine learning model from a source language to the target language using a second dataset, the second dataset comprising healthy speech data in the source and target languages; extracting the feature set from a subject’s speech data provided in the source language; applying the optimal transport mapping on the extracted feature set to provide mapped features; and providing the mapped features as input to the machine learning model to classify the input as impaired speech or healthy speech; and obtaining a classification of the input as impaired speech or healthy speech.

The examples and embodiments are presented only by way of example and are not meant to limit the scope of the subject matter described herein. Individual features of each example or embodiment presented above may be combined, in whole or in part, with individual features of other examples or embodiments. Some steps or acts in a process or method may be reordered or omitted, and features and aspects described in respect of one embodiment may be incorporated into other described embodiments. Variations of these examples will be apparent to those in the art and are considered to be within the scope of the subject matter described herein.

The data employed by the systems, devices, and methods described herein may be stored in one or more data stores. The data stores can be of many different types of storage devices and programming constructs, including but not limited to RAM, ROM, flash memory, programming data structures, programming variables, and so forth. Code adapted to provide the systems and methods described above may be provided on many different types of computer-readable media including but not limited to computer storage mechanisms (e.g., CD-ROM, diskette, RAM, flash memory, computer hard drive, etc.) that contain instructions for use in execution by one or more processors to perform the operations described herein. The media on which the code may be provided is generally considered to be non-transitory or physical.

Computer components, software modules, engines, functions, and data structures may be connected directly or indirectly to each other in order to allow the flow of data needed for their operations. The data processing and computer systems described above may be provided in a single location, or may be distributed in a cloud environment, using techniques known to those skilled in the art. Those skilled in the art will know how to implement the particular examples described above in the systems and methods of FIGS. 1-3. Various functional units have been expressly or implicitly described as modules, engines, or similar terminology, in order to more particularly emphasize their independent implementation and operation. Such units may be implemented in a unit of code, a subroutine unit, object, applet, script or other form of code. Such units may also be implemented in hardware circuits comprising custom circuits or gate arrays; field-programmable gate arrays; programmable array logic; programmable logic devices; commercially available logic chips, transistors, and other such components. As will be appreciated by those skilled in the art, where appropriate, functional units need not be physically located together, but may reside in different locations, such as over several electronic devices or memory devices, capable of being logically joined for execution. Functional units may also be implemented as combinations of software and hardware, such as a processor operating on a set of operational data or instructions.

Use of any particular term should not be construed as limiting the scope or requiring experimentation to implement the claimed subject matter or embodiments described herein. Any suggestion of substitutability of the data processing or computer systems or environments for other implementation means should not be construed as an admission that the invention(s) described herein are abstract, or that the data processing systems or their components are non-essential to the invention(s) described herein.

A portion of the disclosure of this patent document contains material which is or may be subject to one or more of copyright, design, or trade dress protection, whether registered or unregistered. The rightsholder has no objection to the reproduction of any such material as portrayed herein through facsimile reproduction of this disclosure as it appears in the Patent Office records, but otherwise reserves all rights whatsoever.

1-43. (canceled)

44. A method comprising:
obtaining a feature set from a subject’s speech data in a source language;
applying a mapping to the extracted feature set to provide mapped features, wherein the mapping is defined by a domain adaptation system trained on a domain adaptation dataset comprising healthy speech data in the source language and a target language;
providing the mapped features as input to a classifier trained in the target language to classify impaired speech and healthy speech; and
obtaining a classification of the subject’s speech data as impaired speech or healthy speech from the classifier wherein impaired speech is aphasic speech.

45. The method of claim 44, further comprising recording the subject’s speech and transcribing the recorded data, and obtaining the feature set from the transcribed speech.

46. The method of claim 44, wherein the feature set comprises a distribution of parts of speech in the speech data.

47. The method of claim 44, wherein the domain adaptation dataset comprises unpaired data.

48. The method of claim 47, wherein the domain adaptation dataset comprises less than 10% impaired speech data.

49. The method of claim 48, wherein the domain adaptation dataset comprises less than 5% impaired speech data.

50. The method of claim 44, wherein the target language and the source language are dissimilar languages.

51. The method of claim 50, wherein the target language and the source language have different subject, verb, and object ordering.

52. The subject matter of claim 50, wherein the target language and the source language differ in usage of reduplication.

53. The method of claim 50, wherein the target language is English and the source language is Mandarin.

54. The method of claim 44, wherein the domain adaptation system comprises an optimal transport system.

55. The method of claim 44, wherein the optimal transport system employs an Earth Movers Distance, Gaussian Optimal Transport Mapping, or Entropic Regularized Optimal Transport Solver algorithm.

56. The method of claim 44, further comprising providing the domain adaptation system by:
generating a machine learning model for classifying input as impaired speech or healthy speech, the machine learning model trained on a first dataset in the target language, the first dataset comprising impaired speech data and healthy speech data; and
generating an optimal transport mapping of a feature set employed in the machine learning model from the source language to the target language using a second dataset, the second dataset comprising healthy speech data in the source and target languages.

57. Non-transitory computer-readable media storing code which, when executed by one or more processors of a computer system, causes the system to implement:
obtaining a feature set from a subject’s speech data in a source language;
applying a mapping to the extracted feature set to provide mapped features, wherein the mapping is defined by a domain adaptation system trained on a domain adaptation dataset comprising healthy speech data in the source language and a target language;
providing the mapped features as input to a classifier trained in the target language to classify impaired speech and healthy speech; and
obtaining a classification of the subject’s speech data as impaired speech or healthy speech from the classifier wherein impaired speech is aphasic speech.

58. The computer-readable media of claim 57, wherein the system is further caused to implement recording the subject’s speech and transcribing the recorded data, and obtaining the feature set from the transcribed speech.

59. The computer-readable media of claim 57, wherein the feature set comprises a distribution of parts of speech in the speech data.

60. The computer-readable media of claim 57, wherein the domain adaptation dataset comprises unpaired data and less than 10% impaired speech data.

61. The computer-readable media of claim 57, wherein the target language and the source language have different subject, verb, and object ordering.

62. The computer-readable media of claim 47, wherein the domain adaptation system comprises an optimal transport system.

63. A networked computer system comprising:
at least one network communication subsystem;
memory; and
at least one or more processors configured to implement:
obtaining a feature set from a subject’s speech data in a source language;
applying a mapping to the extracted feature set to provide mapped features, wherein the mapping is defined by a domain adaptation system trained on a domain adaptation dataset comprising healthy speech data in the source language and a target language;
providing the mapped features as input to a classifier trained in the target language to classify impaired speech and healthy speech; and
obtaining a classification of the subject’s speech data as impaired speech or healthy speech from the classifier wherein impaired speech is aphasic speech.
